(19) United States
(12) Patent Application Publication
(10) Pub. No.: US 2006/0226251 A1
(43) Pub. Date: Oct. 12, 2006

(54) Diffusion device

(76) Inventors: Thomas A. Helf, New Berlin, WI (US); Edward L. Paas, Los Altos, CA (US); Heather R. Schramm, Whitewater, WI (US); Scott D. Walter, Twin Lakes, WI (US)

Correspondence Address: S.C. JOHNSON & SON, INC., 1525 HOWE STREET, RACINE, WI, 53403-2236, US

(21) Appl. No.: 11/403,166

(22) Filed: Apr. 12, 2006

Related U.S. Application Data
(60) Provisional application No. 60/670,519, filed on Apr. 12, 2005.

(51) Int. Cl. A24F 25/00; A61L 9/04

(52) U.S. Cl. 239/34

(57) ABSTRACT

A diffusion device comprises a housing and first and second containers disposed within the housing and having first and second wicks, respectively, extending therefrom. The diffusion device further includes first and second active materials disposed in the first and second containers, respectively, and first and second piezoelectric elements disposed adjacent tips of the first and second wicks, respectively. Still further, the diffusion device includes a switch disposed on a top surface of the housing, wherein the switch is adapted to control the mode of operation of the device. The device includes first, second, and third modes of operation, wherein in the first mode of operation, the device emits the first active material, in the second mode of operation, the device emits the second active material, and in the third mode of operation, the device alternates between emitting the first and second active materials.

### CROSS REFERENCE TO RELATED APPLICATIONS

This application is based on and claims priority from provisional patent Application No. 60/670,519, filed on Apr. 12, 2005, which is hereby incorporated by reference in its entirety.

### REFERENCE REGARDING FEDERALLY SPONSORED RESEARCH OR DEVELOPMENT

Not applicable

### SEQUENTIAL LISTING

Not applicable

### BACKGROUND

1. Technical Field

The present invention relates to diffusion devices, and more particularly, to diffusion devices for emitting more than one active material therefrom.

2. Description of the Background

A multitude of active material diffusion devices or diffusers exist in the marketplace. Many of such devices are passive devices that require only ambient air flow to disperse the liquid active material therein. Other devices are battery-powered or receive household power via a cord and plug extending from the device.

Various means for dispensing active materials from diffusion devices are also known in the art. For example, some diffusion devices include a heating element for heating an active material to promote vaporization thereof. Other diffusion devices employ a fan or blower to generate air flow to direct active material out of the diffusion device into the surrounding environment. In another type of diffusion device, active material may be emitted from the device using a bolus generator that delivers a pulse of air to eject a scent ring. Still other diffusion devices dispense active materials utilize ultrasonic means to dispense active materials therefrom.

In one example a diffusion device includes two heaters for dispersion of fragrances. The device includes a housing, a plug extending from the housing for insertion into an outlet, and two containers having fragrances therein and wicks extending therefrom to absorb fragrances from the containers. Each of the heaters is disposed adjacent one of the wicks to heat the respective wick to vaporize the fragrances therein. Optionally, a CPU controlled by internal software may first activate a first of the two heaters for a predetermined period of time. After the period of time expires, the CPU deactivates the first heater and thereafter activates the second heater.

Other diffusion devices include a housing having a cavity for receiving a cartridge. The cartridge generally has a plurality of scent elements disposed on a rotatable disk. A blower is mounted in the housing to generate airflow by passing air across a scent element and out an aperture in the housing. The housing further includes rotating means that rotate the rotatable disk, thereby rotating the scent elements thereon. The device diffuses a first scent for a predetermined time period and thereafter rotates the disk to a second scent and diffuses the second scent for the predetermined time period. This process repeats itself until the last scent element is diffused for the time period and then the disk is rotated to a home position.

Piezoelectrically actuated vibratory type liquid atomization apparatuses are described in Helf et al. U.S. Pat. No. 6,293,474, Martin et al. U.S. Pat. No. 6,341,732, Tomkins et al. U.S. Pat. No. 6,382,522, Martens, III et al. U.S. Pat. No. 6,450,419, Boticki et al. U.S. Pat. No. 6,843,430, all of which are assigned to the assignee of the present application and which are hereby incorporated by reference herein. These patents describe a piezoelectrically actuated vibratory type liquid atomization apparatus comprising a piezoelectric actuating element coupled to a liquid atomization plate. The piezoelectric actuating element vibrates the liquid atomization plate in response to alternating electrical voltages applied to the actuating element. The vibration of the plate causes atomization of a liquid supplied to it by a liquid delivery system. An electrical circuit is provided to supply the alternating electrical voltages to conductive elements that are in electrical contract with opposite sides of the actuating element. The conductive elements may also serve to support the actuating element and the liquid atomization plate in a housing that contains the device.

### SUMMARY OF THE INVENTION

According to one aspect of the present invention, a diffusion device comprises a housing and first and second containers disposed within the housing and having first and second wicks, respectively, extending therefrom. The diffusion device further includes first and second active materials disposed in the first and second containers, respectively, and first and second piezoelectric elements disposed adjacent tips of the first and second wicks, respectively. Still further, the diffusion device includes a switch disposed on a top surface of the housing, wherein the switch is adapted to control the mode of operation of the device. The device includes first, second, and third modes of operation, wherein in the first mode of operation, the device emits the first active material, in the second mode of operation, the device emits the second active material, and in the third mode of operation, the device alternates between emitting the first and second active materials.

According to another aspect of the present invention, a combination includes a diffusion device having a housing, first and second containers disposed within the housing, and first and second wicks extending respectively from the first and second containers. The diffusion device further includes first and second active materials disposed in the first and second containers, respectively, and first and second piezoelectric elements disposed adjacent tips of the first and second wicks, respectively, for dispensing the first and second active materials, respectively. The combination further includes a holder having a cavity disposed therein, wherein the diffusion device is place in the cavity to create a decorative object.

According to yet another aspect of the present invention, a method of disposing first and second active materials from a diffusion device includes the step of inserting a battery into the diffusion device. The method further includes the step of selecting an intensity level for dispersion of the active material(s), wherein the intensity level is determined by the time between active material emissions. Still further, the method includes the step of selecting from one of three different modes of operation including emitting the first active material, emitting the second active material, and alternating between emission of the first and second active materials based on a predetermined emission timing.

According to a further aspect of the present invention, a battery-operated diffusion device includes a housing, a batter disposed within the housing for providing power to the diffusion device, and first and second containers disposed within the housing and having first and second wicks, respectively, extending therefrom. The diffusion device further includes first and second active materials disposed in the first and second containers, respectively and first and second piezoelectric elements disposed adjacent tips of the first and second wicks, respectively. Still further, the diffusion device includes a first switch disposed on a top surface of the housing, wherein the first switch is adapted to control the intensity of diffusion and a second switch disposed on a top surface of the housing, wherein the second switch is adapted to control the mode of operation of the device and includes three different modes of operation for operating the diffusion device.

Other aspects and advantages of the device of the present application will become apparent upon consideration of the following detailed description.

### BRIEF DESCRIPTION OF THE DRAWINGS

FIG. 1 is a top perspective view of a diffusion device;

FIG. 2 is a plan view of the diffusion device of FIG. 1;

FIG. 3 is a plan view similar to that of FIG. 2 in which the housing is removed;

FIG. 4 is a front elevational view of the diffusion device of FIG. 1;

FIG. 5 is an elevational view of a first side of the diffusion device of FIG. 1;

FIG. 6 is an elevational view of a second side of the diffusion device of FIG. 1;

FIG. 7 is back elevational view of the diffusion device of FIG. 1;

FIG. 8 is a bottom perspective view of the diffusion device of FIG. 1;

FIG. 9 is a bottom plan view of the diffusion device of FIG. 1;

FIG. 10 is a bottom plan view similar to that of FIG. 9 in which the bottom cover is removed;

FIG. 11 is a top perspective view of the diffusion device of FIG. 1 disposed within a decorative holder;

FIG. 12 is a top perspective view similar to that of FIG. 11 in which the diffusion device of FIG. 1 is disposed within an alternative decorative holder; and

FIG. 13 is a perspective view similar to that of FIGS. 11 and 12 in which the diffusion device of FIG. 1 is disposed within yet an alternative decorative holder.

FIGS. 14A-14E, when joined along the dotted lines as shown by FIG. 14, are schematic diagrams illustrating an exemplary circuit for controlling one or more components of the diffusion device of FIG. 1; and

FIG. 15 is a flow diagram illustrating the logic associated with switches for controlling the diffusion device of FIG. 1.

### DETAILED DESCRIPTION OF THE PREFERRED EMBODIMENTS

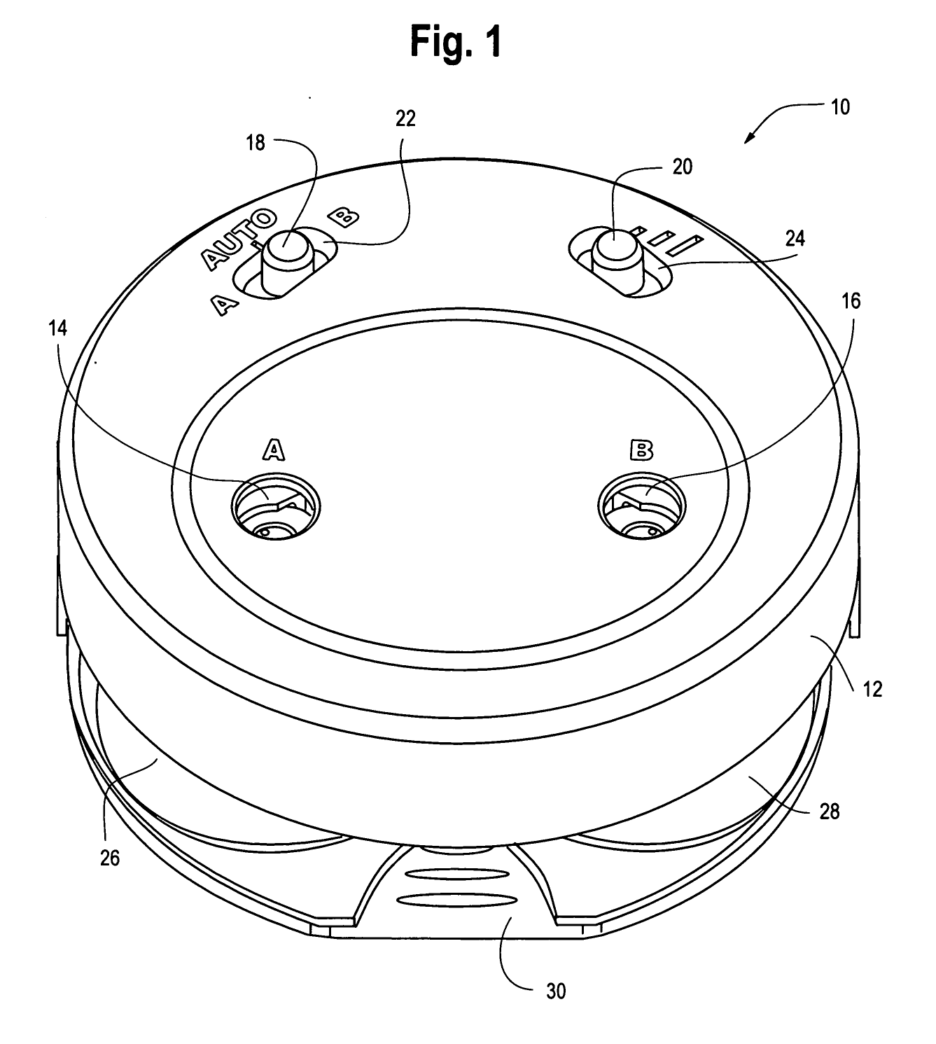

As seen in FIG. 1, a diffusion device 10 includes a cylindrical housing 12. The housing 12 includes two apertures 14 and 16 through which an aerosol active material may be emitted. Two multi-position switches 18 and 20 are disposed within housing 12. An operating mode switch 18 controls the operating mode of diffusion device 10 and extends through another aperture, 22 in the housing 12. An emission frequency switch 20 controls the emission frequency of diffusion device 10 and extends through yet another aperture 24 in the housing 12.

A container 26 containing an active material and having a wick extending therefrom is disposed within the housing 12 and an opening (not shown) of the container 26 is adjacent the aperture 14. An additional container 28, also containing an active material, which may be the same or different than the active material in the container 26, and having a wick extending therefrom, is disposed within the housing 12 and an opening (not shown) of the container 28 is disposed adjacent the aperture 16. The containers 26 and 28 are replaceable.

Illustratively, the types of liquid active materials described herein may be, for example, an insecticide, an insect repellant, an insect attractant, a disinfectant, a mold or mildew inhibitor, a fragrance, a disinfectant, an air purifier, an aromatherapy scent, an antiseptic, an odor eliminator, a positive fragrancing active material, an air-freshener, a deodorizer, or the like, and combinations thereof. The present application contemplates the use of the same or different active materials and/or the same or different types of active materials. For example, both of the containers 26 and 28 may include a lavender fragrance therein. Alternatively, the container 26 may include a strawberry fragrance and the container 28 may include a vanilla fragrance. Still alternatively, the container 26 may include an insect repellant and the container 28 may include an odor eliminator. As such, any combination of types of liquid active materials may be utilized in the containers 26, 28.

A bottom cover 30 is connected to the housing 12 and provides a planar bottom surface for the diffusion device 10. Both the housing 12 and the bottom cover 30 may be made of a thermoplastic material and may be injection molded, although the housing 12 and the bottom cover 30 may be made of any other suitable material.

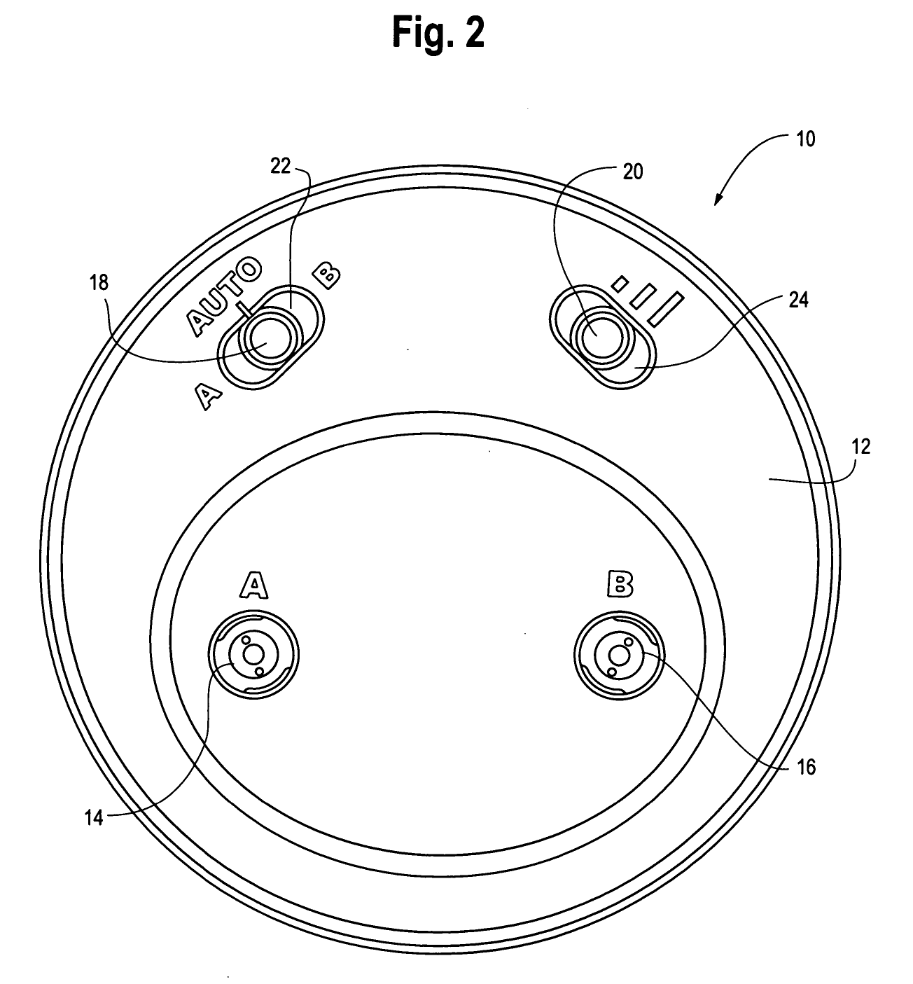

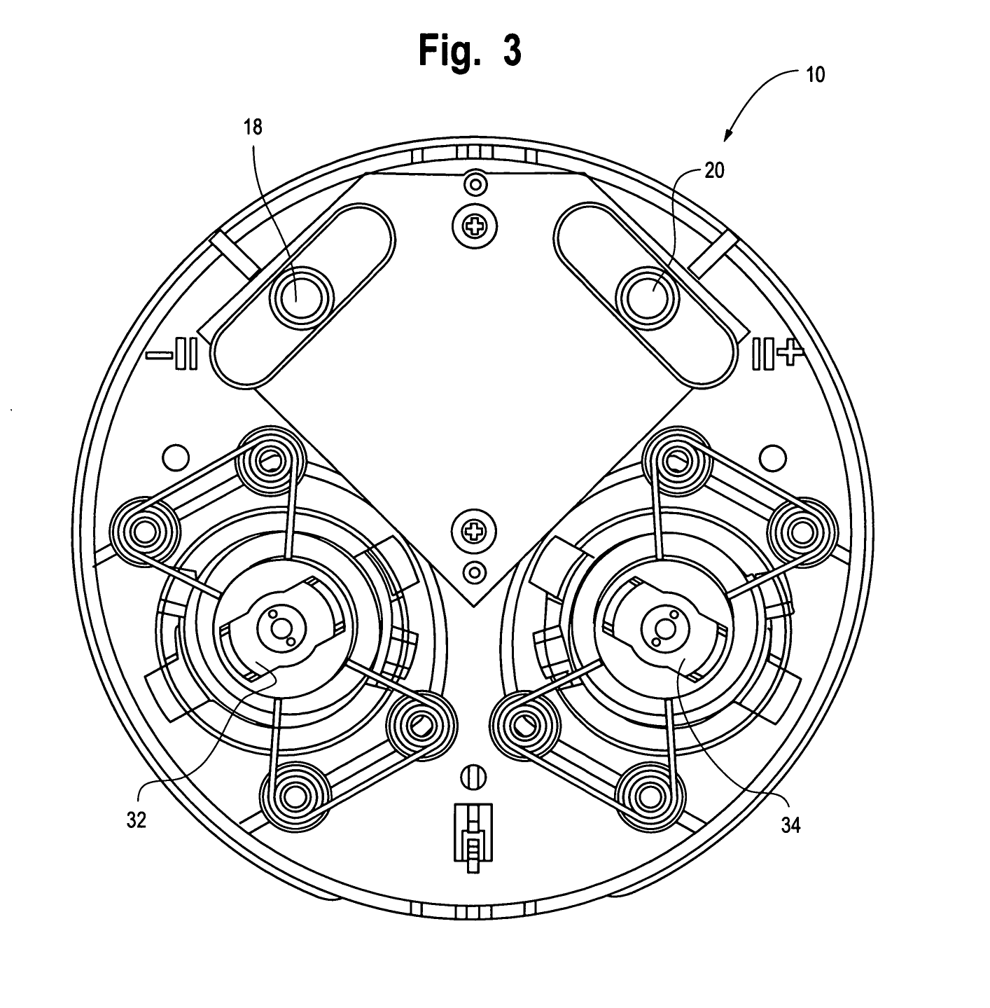

FIGS. 2 and 3 depict top views of the diffusion device 10, wherein like reference numerals throughout the drawings designate like structures. FIG. 3 is similar to FIG. 2 except that the housing 12 has been omitted in FIG. 3. The operating mode switch 18 and the emission frequency switch 20 are shown in FIG. 3, as are the piezoelectric devices 32 and 34. The use of piezoelectric devices to atomize liquids is well known; and examples of such devices are described in Martens, III et al. U.S. Pat. No. 6,450,419, Helf et al. U.S. Pat. No. 6,706,988, and Boticki et al. U.S. Pat. No. 6,843,430 incorporated by reference herein. In general, these devices apply an alternating voltage to a piezoelectric element to cause the element to expand and contract. The piezoelectric element is coupled to a perforated orifice plate, which in turn is in surface tension contact with a liquid source. The expansion and contraction of the piezoelectric element causes the orifice plate to vibrate up and down whereupon liquid is driven through the perforations and is then emitted upwardly in the form of aerosolized particles.

The piezoelectric device 32 is located within the housing 12 between the container 26 and the aperture 14 and works as described above to atomize the active material of the container 26. Similarly, the piezoelectric device 34 is located within the housing 12 between the container 28 and the aperture 16 within the housing 12 and also works as described above to atomize the active material of the container 28.

The operating mode switch 18 controls the mode of operation of the diffusion device 10. For example, in one embodiment, the operating mode switch 18 may be a slide switch with three different positions. When a user slides the switch 18 to a first position, a mode “A” of operation may be initiated wherein a first active material is emitted continuously at a selected intensity level. When the user slides the switch 18 to a second position, a mode “B” of operation may be entered wherein a second active material is emitted continuously at a selected intensity level. When the switch 18 is moved to a third position, an “auto” mode of operation may be initiated wherein the diffusion device 10 alternates between emitting the first active material and the second active material. Illustratively, in the third mode, the first active material may be emitted for a predetermined period of time and, when the predetermined period of time has expired, the second active material may be emitted for a predetermined period of time that may or not be the same as that of the first active material. The predetermined periods may be any preferred periods of time, but preferably are between about one minute and about twenty-four hours. In one preferred embodiment, the predetermined period is three hours. In another preferred embodiment, the predetermined period is twenty-four hours. In yet another preferred embodiment, the predetermined period is sixty minutes. Still further, in another preferred embodiment, the predetermined period is ninety minutes.

Optionally, additional and/or substitute modes of operation may be used with the device 10 of the present application. Such modes of operation may be utilized with changes to the circuitry and/or additional circuitry. Illustratively, one mode varies the output of the active material(s) from the diffusion device 10. For example, the output may be varied by gradually increasing or decreasing the amount of active material emitted by the device. Optionally, the amount of active material may be increased to a higher amount or level of active material and may remain at that level for a predetermined period of time. The predetermined period of time may be any time limit that prevents habituation of the active material, such as any time period between one minute and thirty minutes. After the predetermined period of time, the amount of active material emitted may be decreased to a lower level and may remain at that level for the same or a different predetermined period of time. This cycle may be repeated continuously or may be repeated in a random or complex pattern. Also, any number of different active material emission levels may be utilized in such a mode of operation.

In another mode of operation, emission of active material may be discontinued for a predetermined period of time. The predetermined period of time may be any period of time that allows the active material level to decrease or partially or fully dissipate from the surrounding environment, but preferably the predetermined time period is between about one minute and about thirty minutes. After the predetermined time has expired, the emission of active material is resumed. This cycle may be repeated with the same, increasing, or decreasing periods of time. Still alternatively, in another mode of operation, two or more active materials may be dispensed simultaneously.

Any of the modes of operation as disclosed herein or as known in the art may be utilized alone or in any combination. Also, any of these modes of operation may be utilized with a diffusion device that emits a single active material or a diffusion device that emits multiple active materials.

The emission frequency switch 20 controls the frequency of active material emission of the diffusion device 10. For example, in one embodiment, the switch 20 may be a slide switch with three different positions. A first position may actuate a dwell period of a first predetermined period of time, wherein the dwell period represents a duration between sprays in which diffusion device 10 is inactive, i.e., not emitting active material. A second position may actuate a dwell period of a second predetermined period of time. A third position may actuate a dwell period of a third predetermined period of time. The predetermined time periods may be of preferred durations, but preferably are between a few seconds and a few minutes. Most preferably, the first, second, and third predetermined time periods are nine seconds, twelve seconds, and eighteen seconds, respectively.

Optionally, a slide switch with five different positions may be utilized, wherein the dwell periods may be similar to those of the slide switch with three different positions, but are preferably between a few seconds and a few minutes. Still optionally, the switches 18 and 20 may include any number of positions corresponding to a preferred number of modes or intensities.

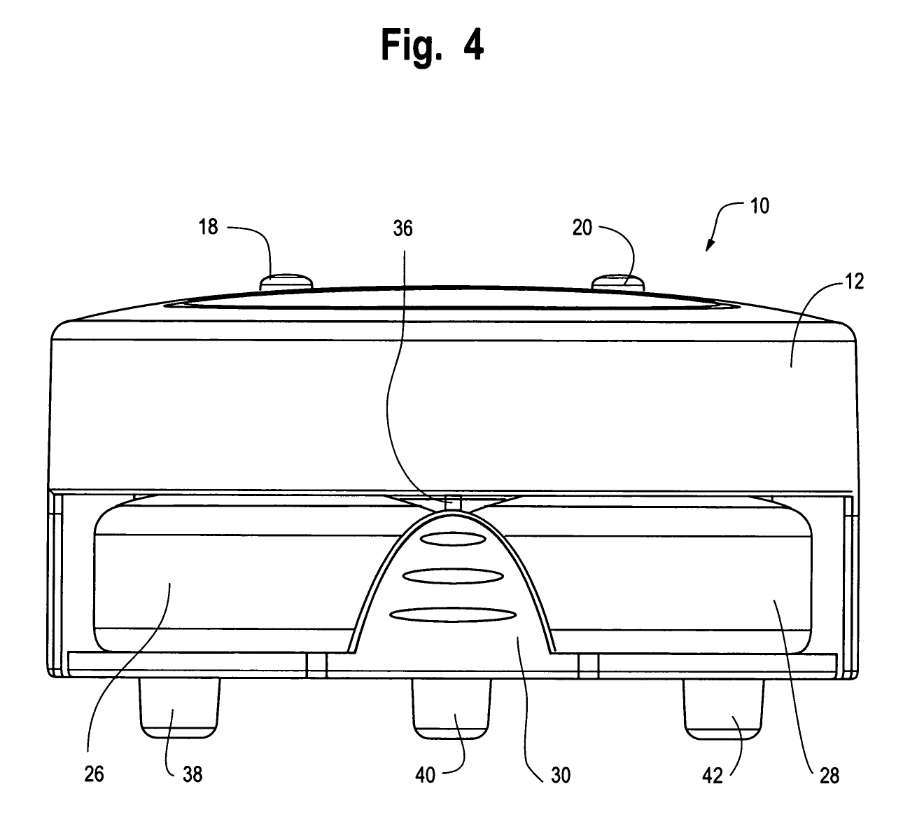

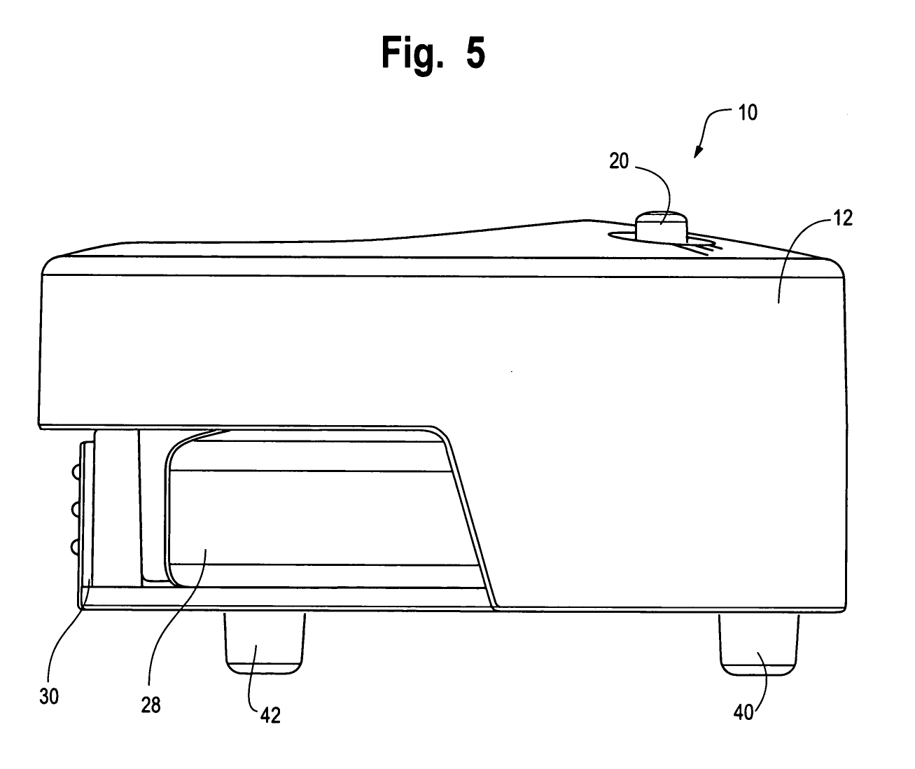

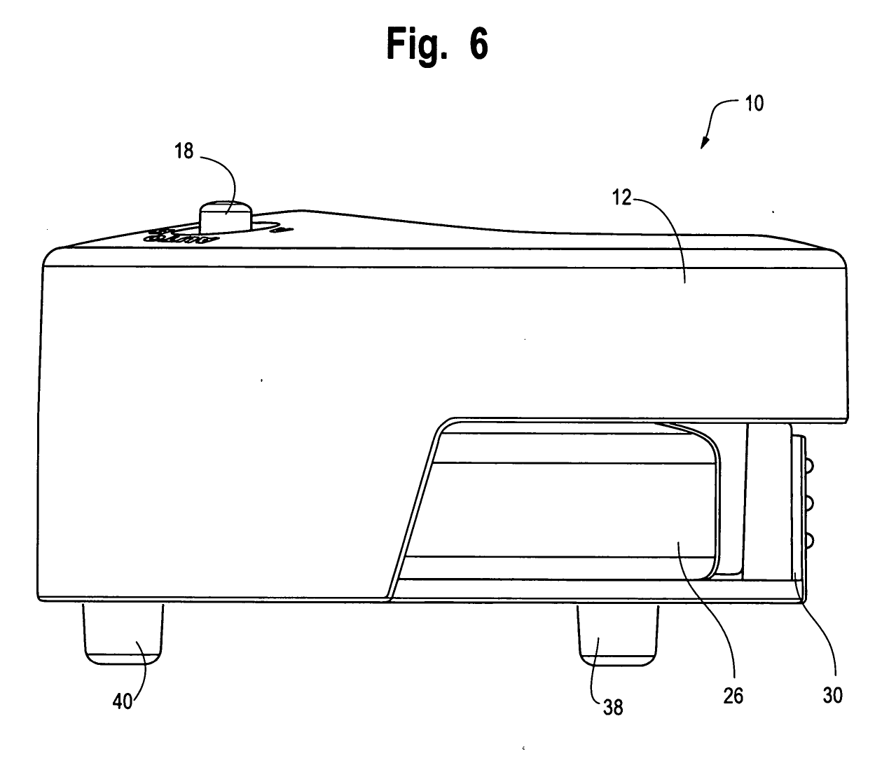

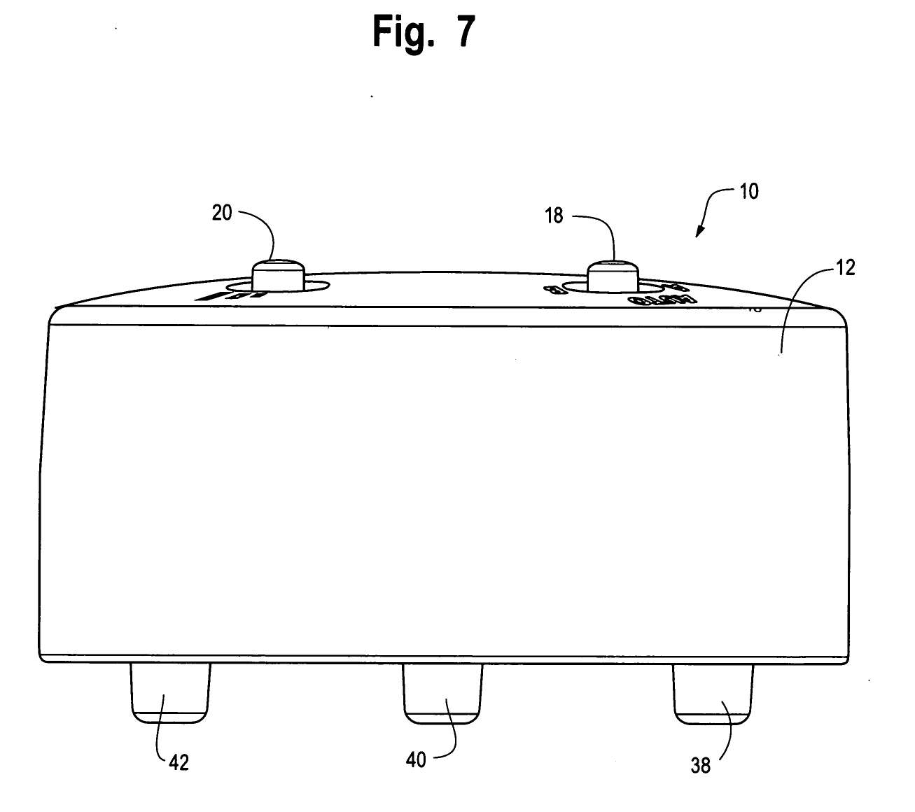

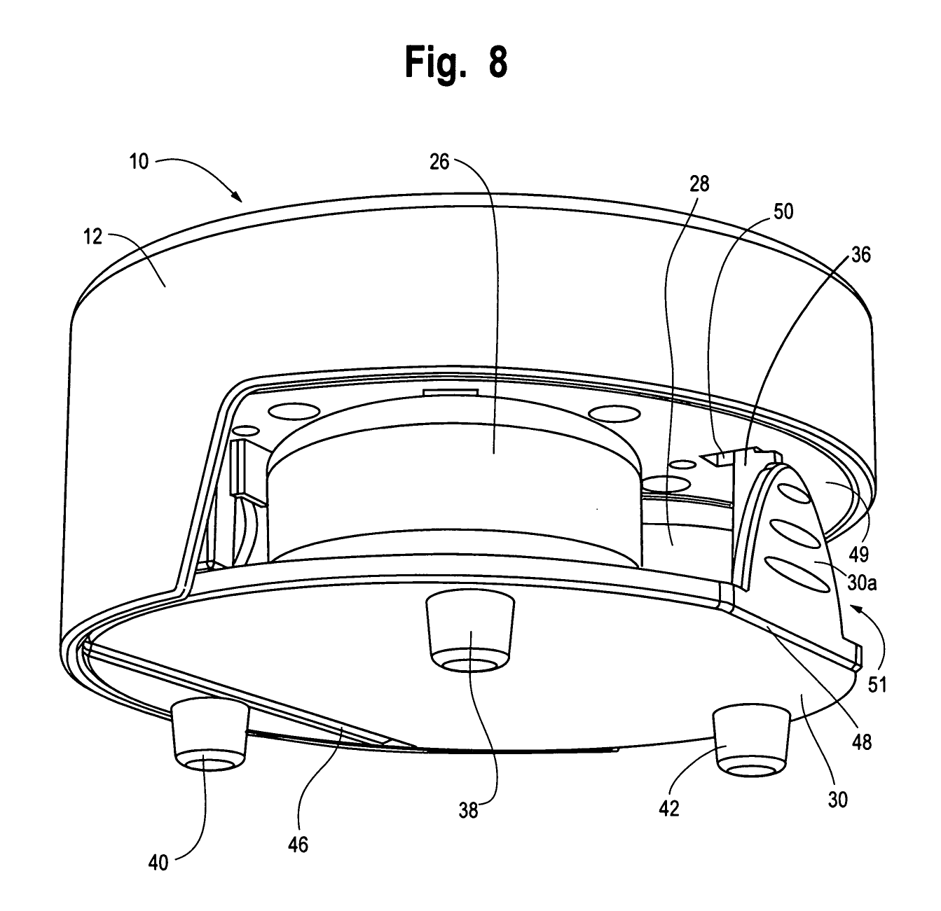

FIGS. 4-8 depict alternative views of the diffusion device 10, and further show the bottom cover 30 and optional legs 38, 40, and 42. Although three legs are depicted, any suitable number of legs that allows the device 10 to stand upright is possible. As shown in FIG. 8, the bottom cover 30 includes two hinged portions 46 and 48. The bottom cover 30 is attached to the housing 12 at a back side of the diffusion device by heat-staking or any other suitable fastening means, including, for example, rivets, press fit, snap fit, screws, ultrasonic welding, adhesives, or the like and combinations thereof. The optional legs 38, 40, and 42 may be attached to the bottom cover 30 in a similar fashion and may be made of a thermoplastic material or any other suitable material.

A flap portion 30a (FIG. 8), extending perpendicularly from the bottom cover 30 at the hinged portion 48 further includes a latch 36 for securing the bottom cover 30 to the housing 12. The latch 36 is adapted to engage a wall or surface 49 defining in part an aperture 50 within the housing 12 and may be flexibly released by pushing the flap portion 30a inward so that the latch 36 may moved out of interfering relationship with the wall or surface 49. The bottom cover 30 may then be lifted away from the housing 12 as it flexes at the hinged portion 46.

As seen in FIGS. 4-6 and 8, when the latch 36 is engaged with the aperture 50 in the wall 49, an opening 51 is formed between the bottom cover 30 and the wall 49. The opening 51 allows a user to determine a fluid level of the active materials in each of the containers 26, 28 without disengaging the latch 36 from the opening 51.

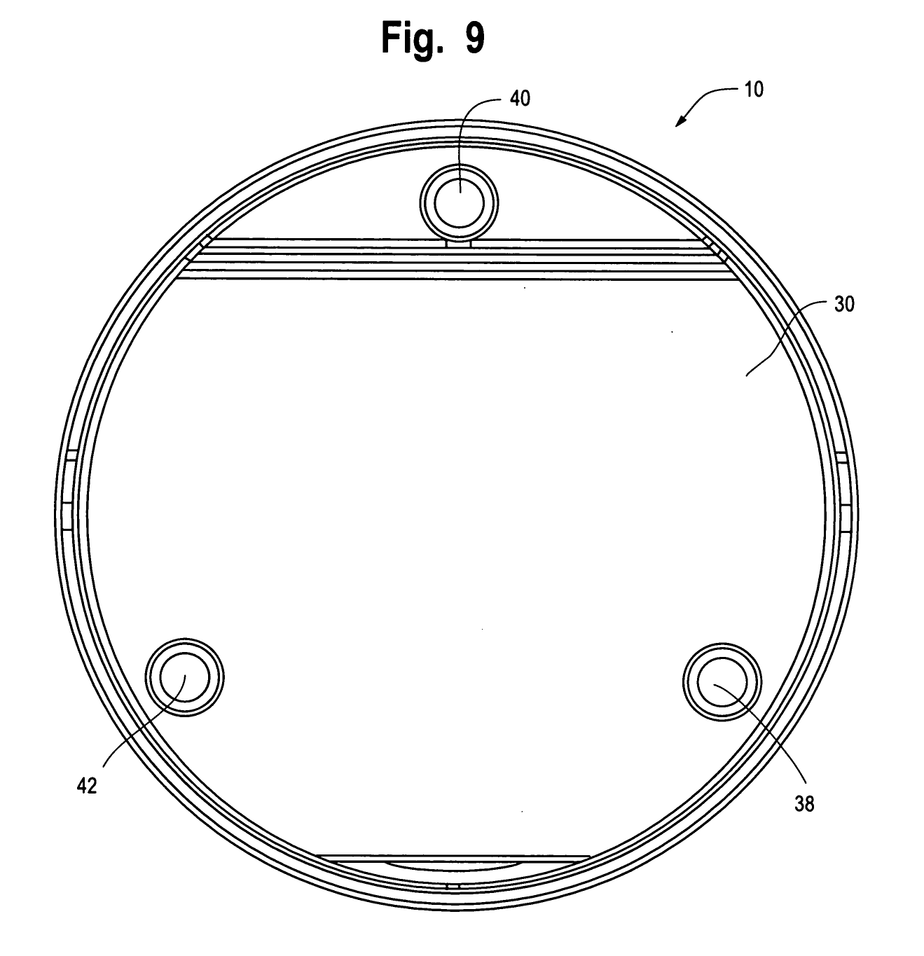

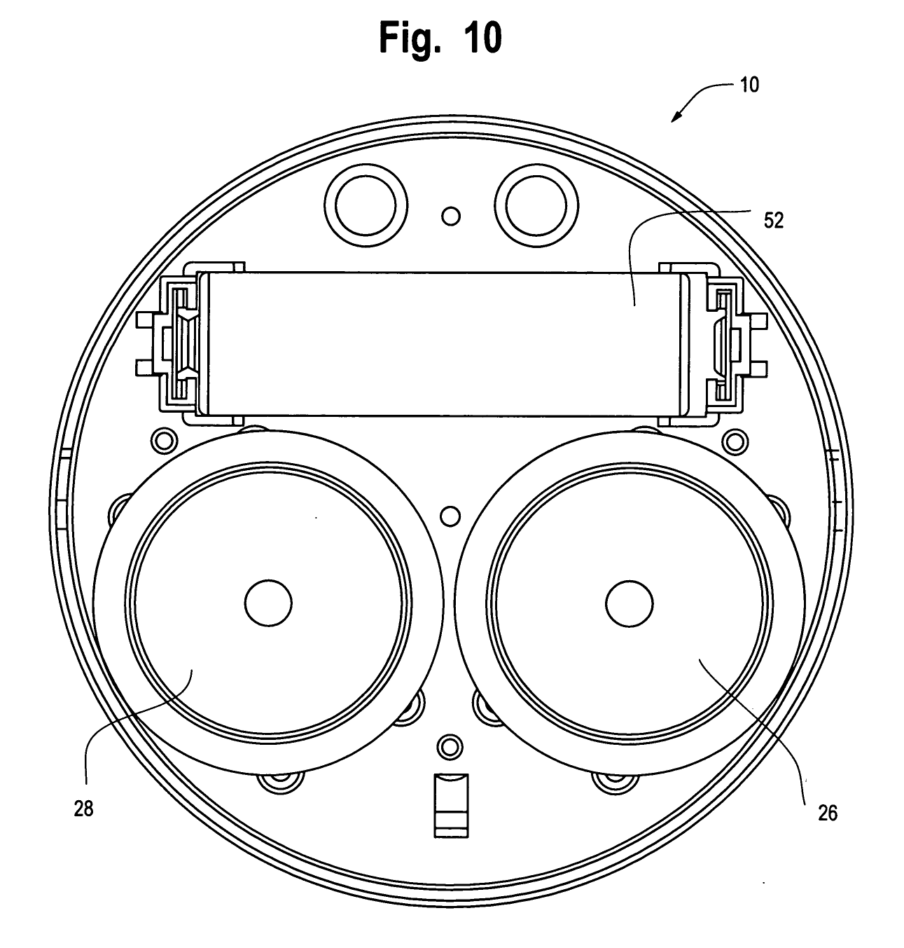

FIGS. 9 and 10 depict bottom views of the diffusion device 10. FIG. 10 is similar to FIG. 9 except that bottom cover 30 has been removed from FIG. 10. The diffusion device includes a battery 52, which provides direct current to the piezoelectric devices 32 and 34. The battery 52 may be any conventional dry cell battery such as “A”, “AA”, “AAA”, “C”, and “D” cells, button cells, watch batteries, and solar cells, but preferably, battery 52 is a “AA” or “AAA” cell battery. Optionally, the diffusion device 10 may be powered by alternating current.

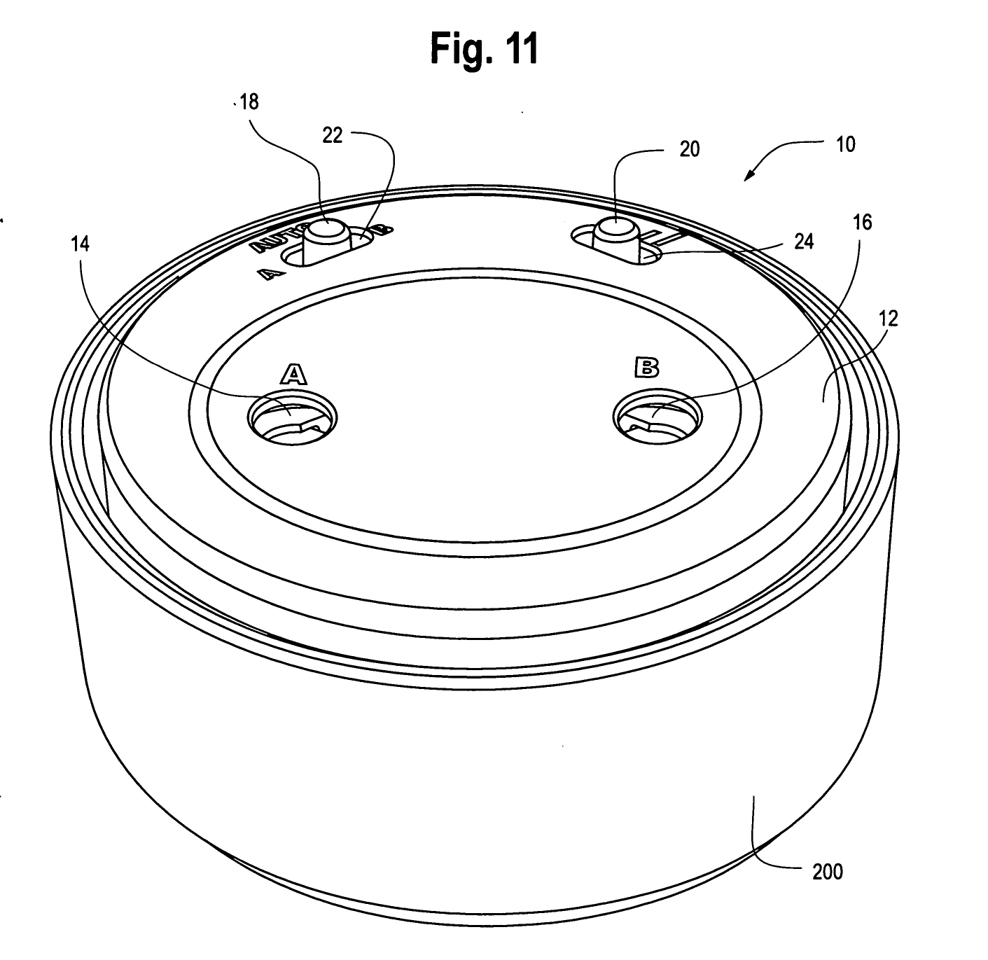

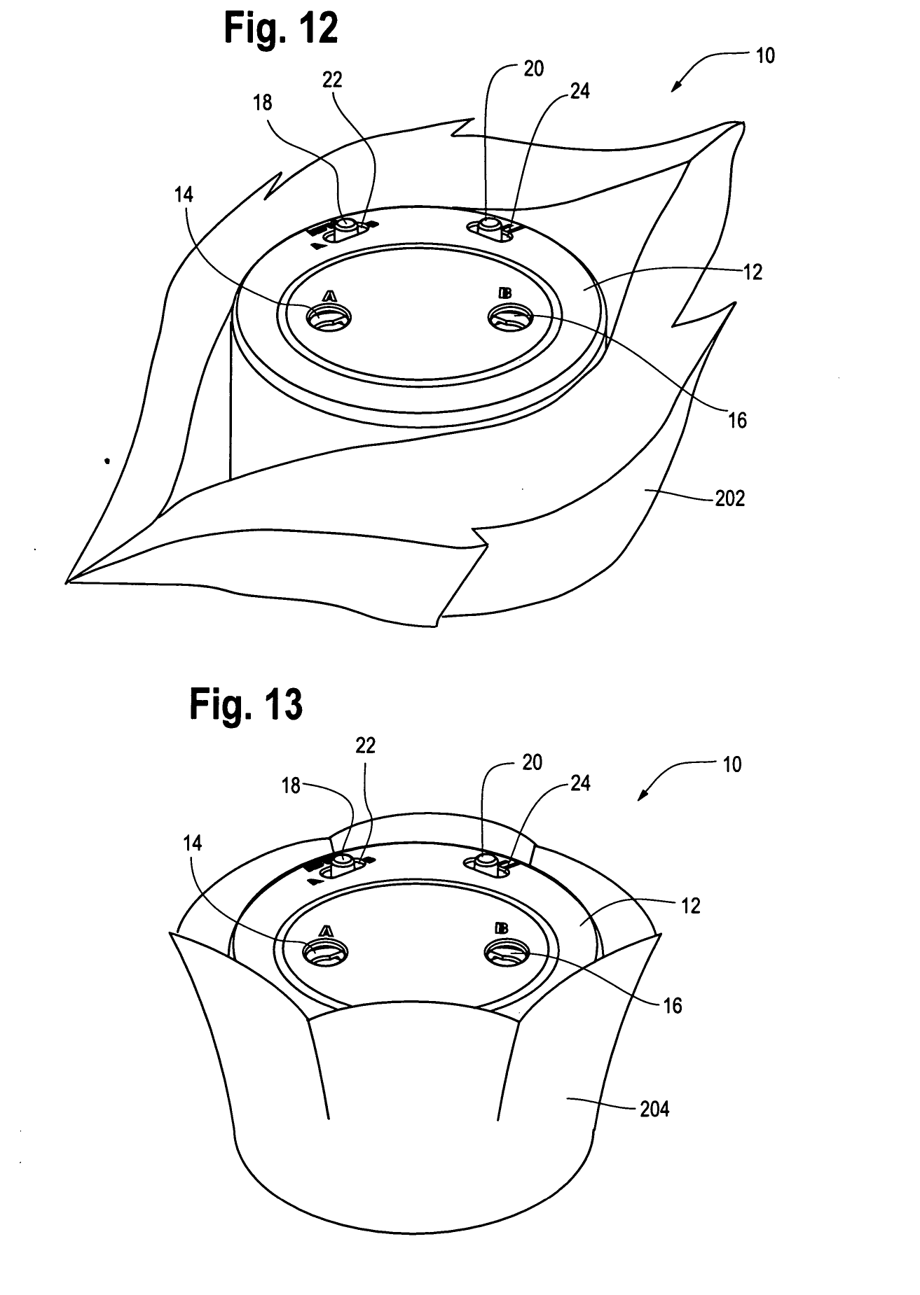

The housing 12 of the diffusion device 10 is preferably generally right circular cylindrically shaped and unadorned, i.e., the housing 12 has a plain, smooth, and regular shape and can be any desired size, but is preferably about 4 inches (10.16 cm) in diameter and is about 2.5 inches (6.35 cm) tall. As shown in FIGS. 11-13, the diffusion device 10 may be disposed within any of numerous decorative holders. As illustrated in FIG. 11, diffusion device 10 may be placed within a cavity of a cylindrical shaped decorative holder 200. Alternatively, diffusion device 10 may be placed within a cavity of a leaf shaped decorative holder 202 as seen in FIG. 12. Yet alternatively, diffusion device 10 may be placed within flower shaped decorative holder 204 as seen in FIG. 13. Alternatively, the decorative holder 200 may be shaped like a pillar candle having the same number of pillars as containers for active materials. Still alternatively, the decorative holder may be shaped like a heart, an animal, a toy, a symbol, or any decorative object.

The decorative holders 200, 202, and 204 are given as illustrations only, as contemplated decorative holders may be of any shape or size and may have any desired design or ornamentation on the exterior and interior surfaces thereof. In addition, such decorative holders may be made from any suitable material including, for example, glass, ceramic and/or plastic such as, for example, nylon, polypropylene, polystyrene, acetal, toughened acetal, polyketone, polybutylene terephthalate, high density polyethylene, polycarbonate, and/or ABS, and combinations thereof.

Optionally, the diffusion device 10 may simply be placed in a decorative holder 200, 202, or 204. In other embodiments, the diffusion device 10 and/or the holder 200, 202, 204 may include means for securing the diffusion device 10 within the holder 200, 202, 204. For example, the diffusion device 10 may be held within the holder 200, 202, 204 by an interference fit therebetween, a frictional fit therebetween, or attachment means may be disposed on one or both of the diffusion device 10 and/or holder 200, 202, 204. Such attachment means may include adhesive tape, hook and loop fasteners, adhesive, or any other attachment means known in the art.

Optionally, the piezoelectric-type diffusers as disclosed herein may be replaced by any other known diffuser. For example, the piezoelectric devices may be replaced by heated-wick type devices, passive devices, aerosol device, and the like and combinations thereof.

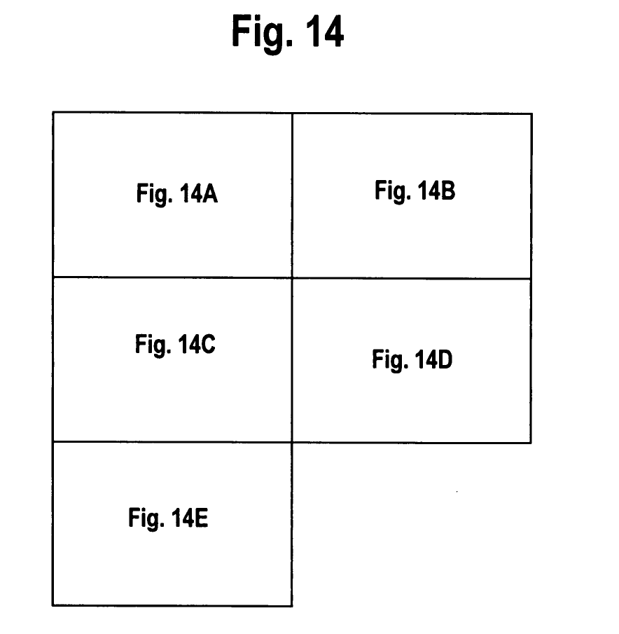

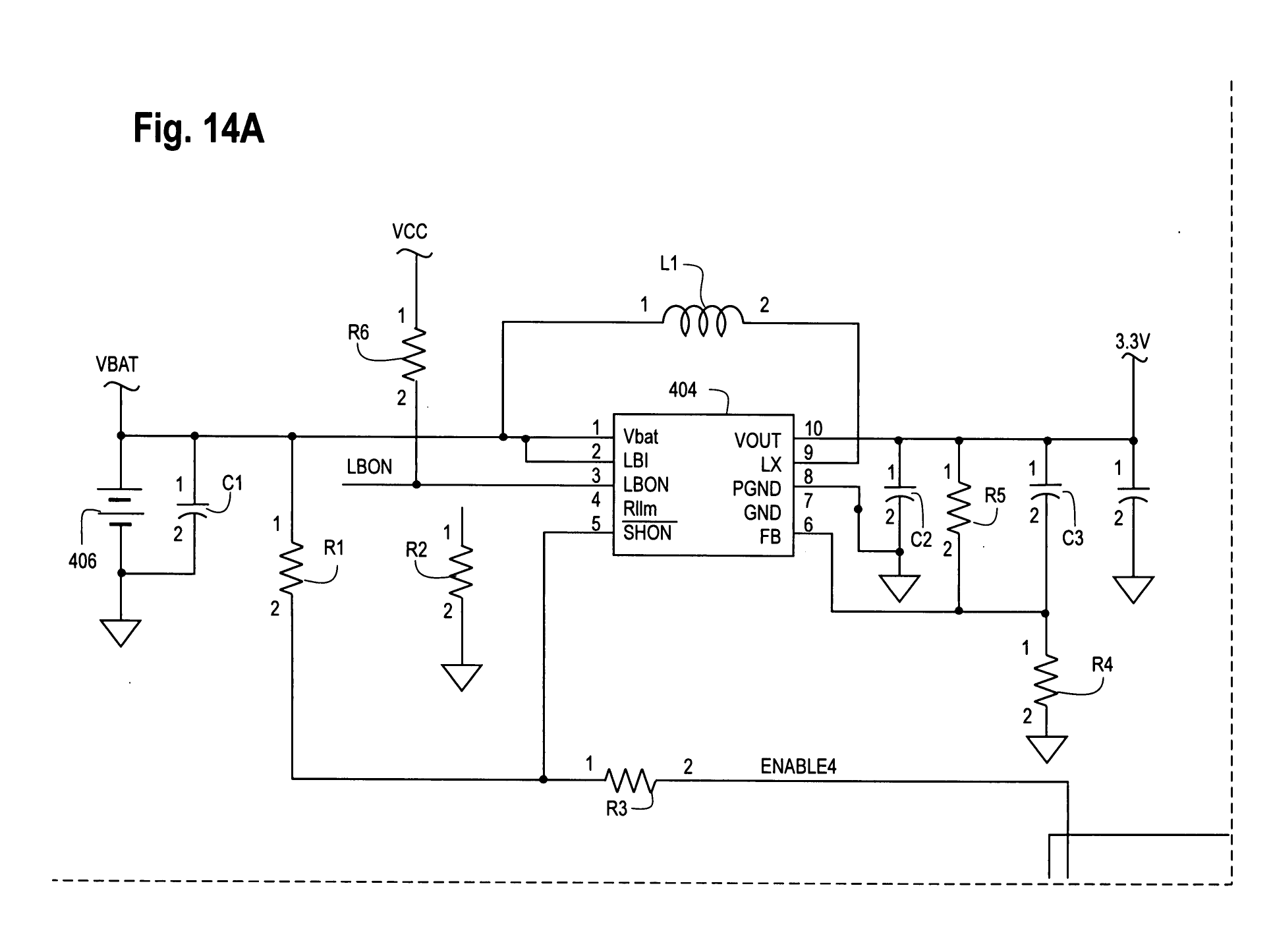

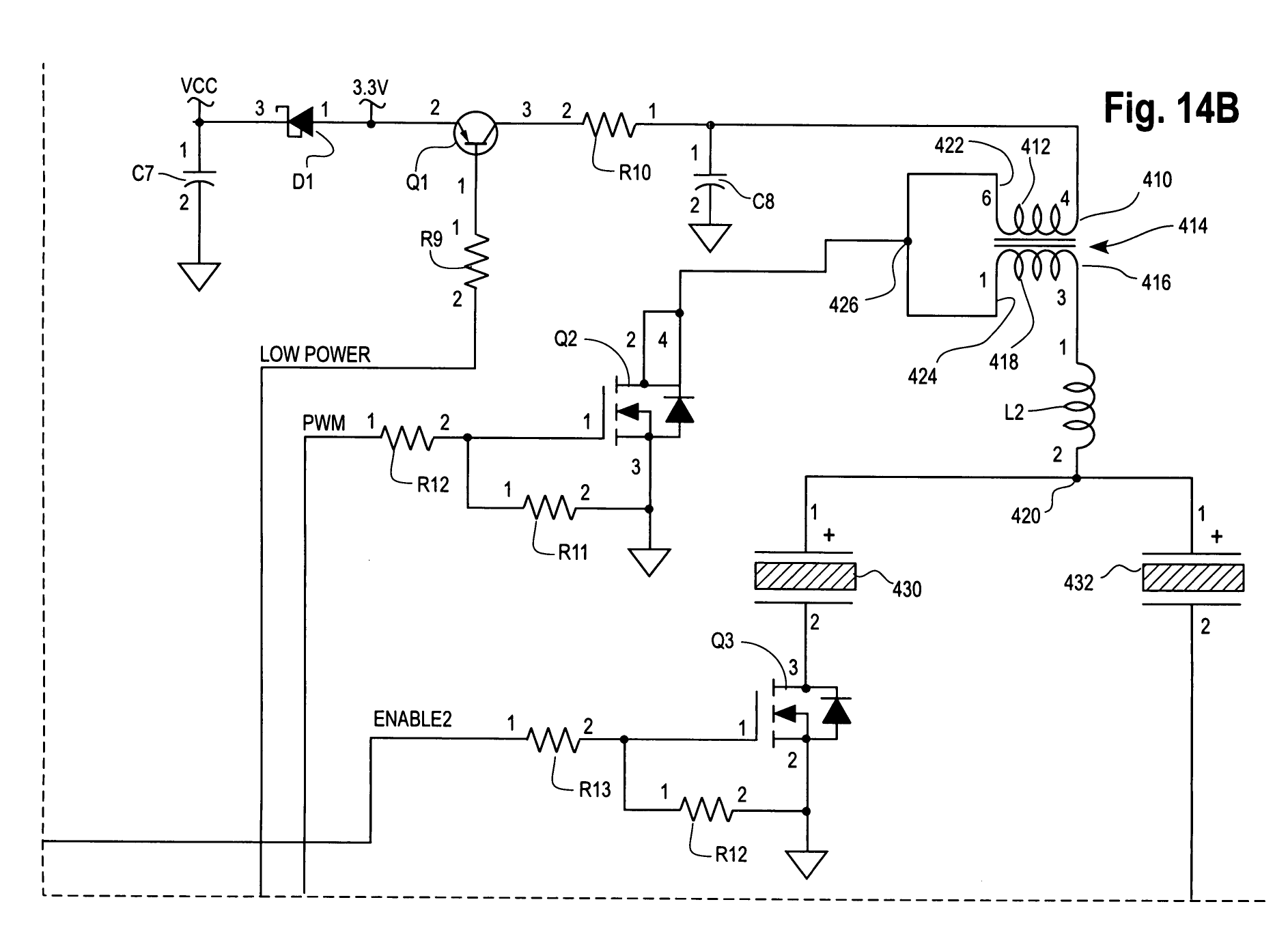

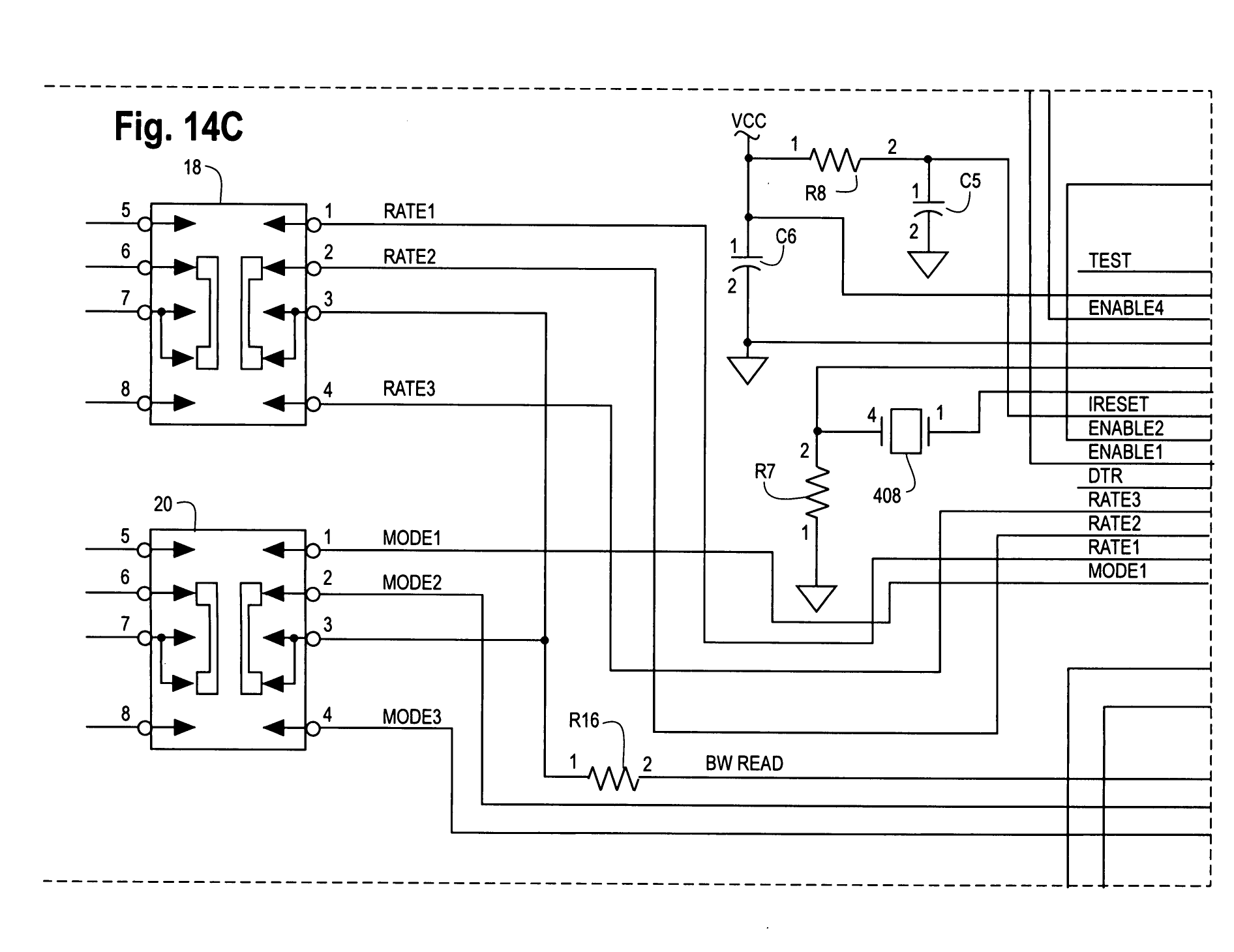

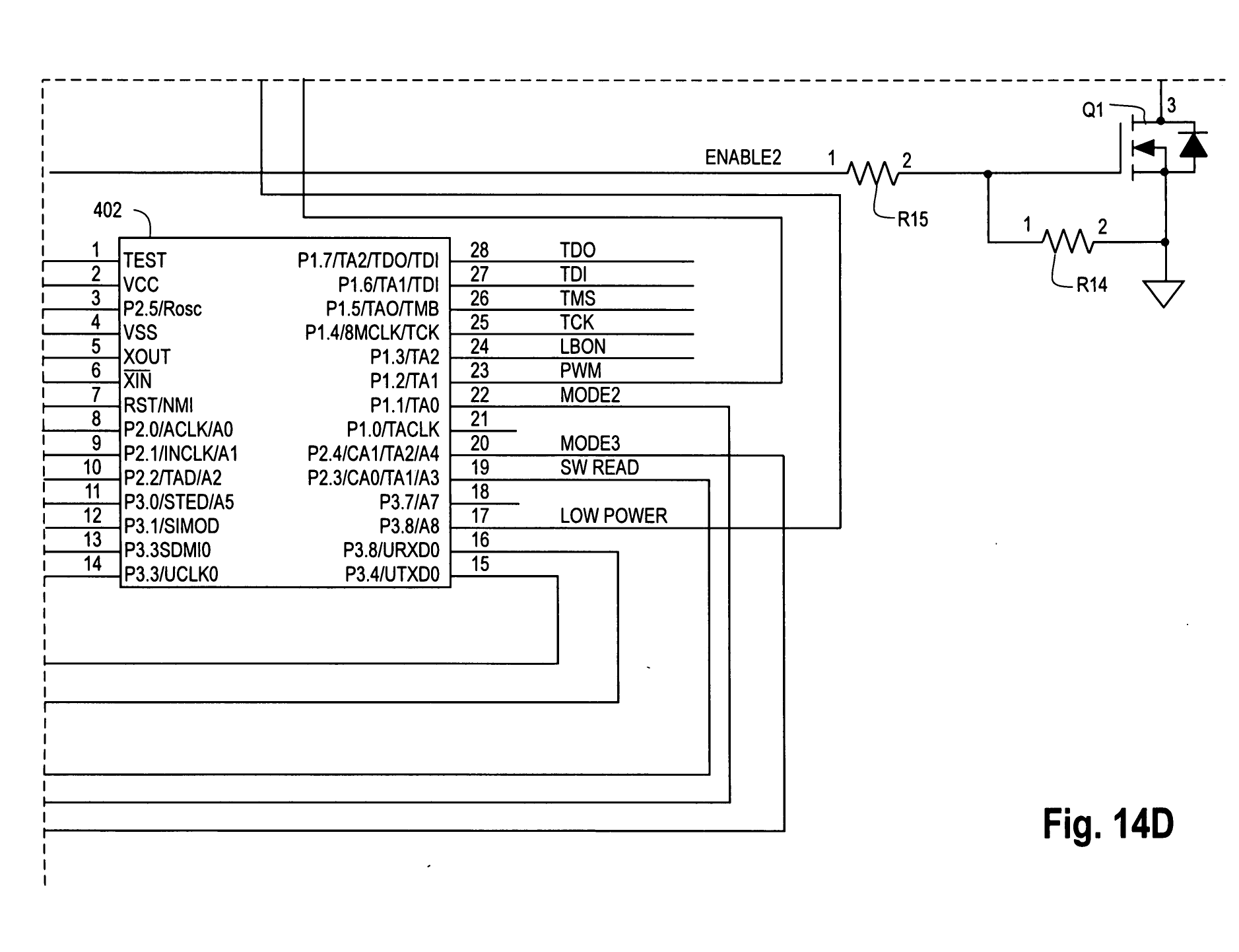

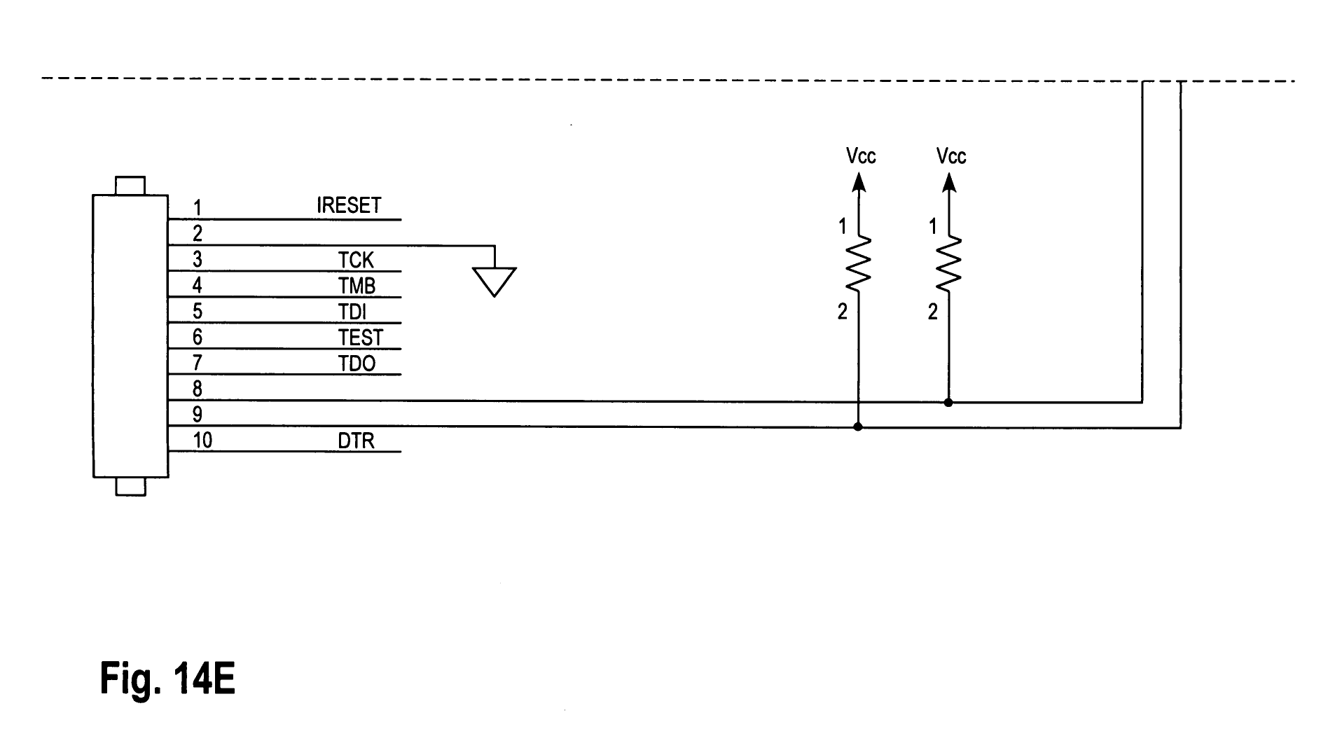

Referring next to FIGS. 14 and 14A-14E, circuitry 400 for operating the device 10 in accordance with a selected mode and selected emission frequency includes a first integrated circuit 402, which may be an application specific integrated circuit (ASIC) or a microprocessor, and a further integrated circuit 404, preferably a high efficiency boost regulator. The IC 402 may comprise an MSP430F122 integrated circuit manufactured by Texas Instruments of Dallas, Tex., whereas the integrated circuit 404 may comprise an SP6648 manufactured by Sipex Corporation of Milpitas, Calif. The integrated circuit 404 receives battery power from a AA size battery 406 and develops supply voltages V_{cc} and a 3.3 volt reference level in conjunction with resistors R1-R6, capacitors C1-C4, and inductor L1.

A pin 3 of the IC 404 is coupled to a pin 24 of the IC 402 for signaling a low-battery condition and a signal ENABLE4 is coupled to a pin 3 and V_{bat} of the IC 402 to ensure normal operation.

The IC 402 includes an internal oscillator that is controlled by a crystal 408 coupled between pins 5 and 6 of the IC 402. A resistor R7 is coupled between one end of the crystal 408 and ground potential. In addition, the IC 402 receives the voltage V_{cc} and ground potential at pins 2 and 4 thereof, respectively. A pin 7 of the integrated circuit 402 is coupled to a junction between a resistor R8 and a capacitor C5. A further end of the resistor R8 is coupled to V_{cc} and a capacitor C6 is coupled between V_{cc} and ground. The IC 402 receives a signal SW_READ at a pin 19 thereof via a resistor R16. The signal SW_READ indicates the positions of the switches 18 and 20. More specifically, the signal SW_READ indicates which of pins 13, 12, and 11 (RATE1, RATE2, and RATE3, respectively) is coupled to pin 19 of the IC 402. Further, SW_READ indicates which of pins 14, 20, and 22 (MODE1, MODE2, and MODE3, respectively) are coupled to pin 19 of the IC 402. The signal SW_READ may be read in conjunction with signals RATE1, RATE2, and RATE 3 and signals MODE1, MODE2, and MODE3.

The IC 402 develops a signal LOW_POWER that is delivered through a resistor R9 to the base of a transistor Q1. An emitter of the transistor Q1 receives the 3.3 volt reference. This helps control the charge current delivered to C8 through R10 from the collector of Q1. A Schottky diode D1 is coupled between the emitter of Q1 and V_{cc}. A further capacitor C7 is coupled between V_{cc} and ground potential. Capacitor C6 is connected to a first terminal 410 of a primary winding 412 of a transformer 414. A first terminal 416 of a secondary winding 418 of the transformer 414 is coupled through an inductor L2 to a junction 420. Second terminals 422 and 424 of the primary and secondary windings 412, 418, respectively are coupled to a further junction 426. The junction 426 is coupled by a transistor 22 to ground. A biasing resistor R11 is coupled between gate and source electrodes of the transistor Q2 and the gate electrode receives a control signal PWM through a resistor R12. The signal PWM is developed at a pin 23 of the IC 402.

The junction 420 is coupled to first terminals of piezoelectric elements 430, 432. The piezoelectric element 430 comprises the driving element for the piezoelectric device 32 whereas the piezoelectric element 432 comprises the driving element for the piezoelectric device 34. Second terminals of the piezoelectric elements 430, 432 are coupled by transistors Q3 and Q4, respectively, to ground. A biasing resistor R12 is coupled between the gate and source electrodes of the transistor Q3 and the gate electrode of the transistor Q3 receives a control signal ENABLE1 through a resistor R13. Similarly, a biasing resistor R14 is coupled between the gate and source electrodes of the transistor Q4 and a control signal ENABLE2 is coupled through a resistor R15 to the gate electrode of the transistor Q4. The control signals ENABLE1 and ENABLE2 are developed at pins 9 and 8, respectively, of the IC 402.

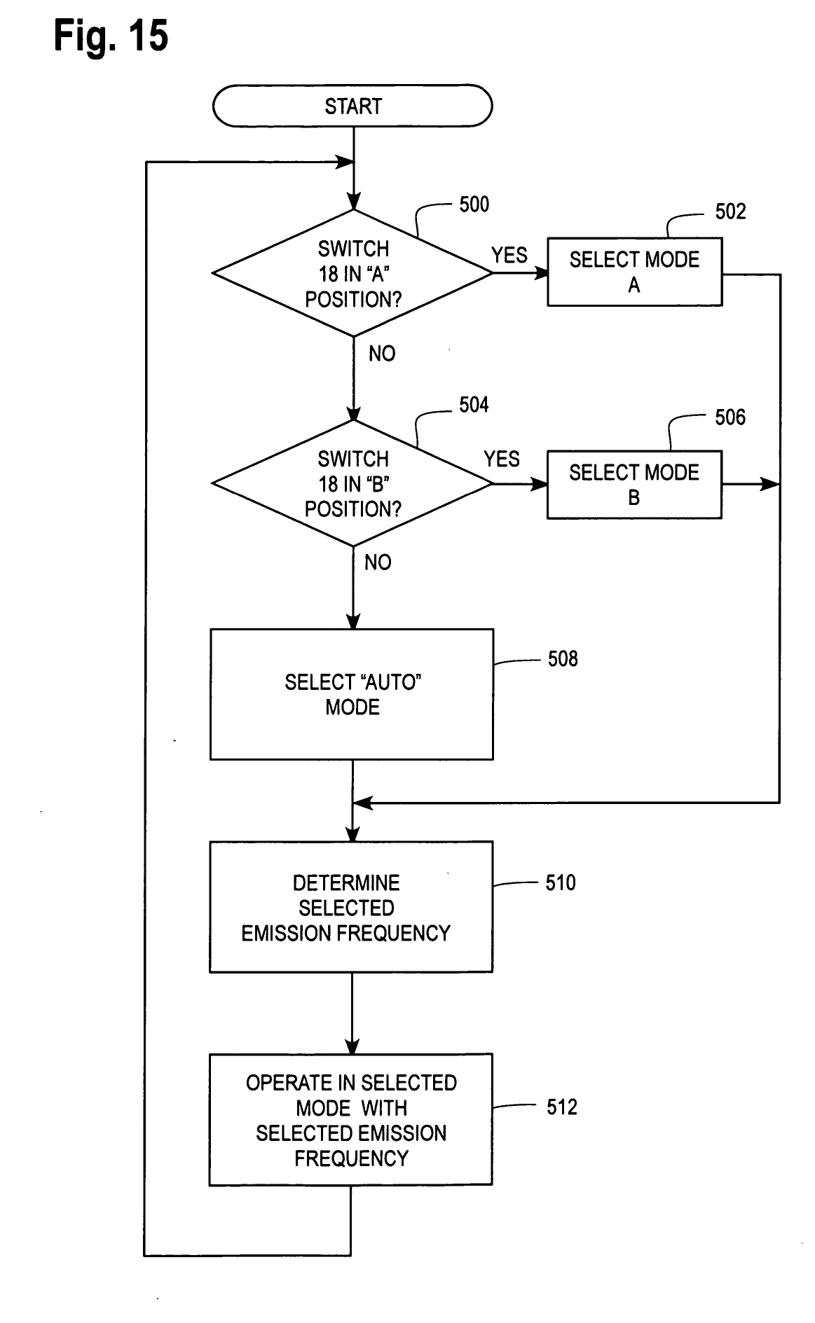

Referring next to the flow chart of FIG. 15, the IC 402 is programmed to cause the device 10 to operate in accordance with a selected mode and emission frequency. As seen in FIG. 15, operation commences at a block 500 which checks to determine whether the switch 18 is in the first position (position “A”). If this is found to be the case, control passes to a block 502 that selects mode A for operation. On the other hand, if the block 500 determines that the switch 18 is not in the first position, then a block 504 checks to determine whether the switch 18 is in the second position (“B” position). If this is the case, then a block 506 selects a mode B of operation. If the block 504 determines that the switch 18 is not in position “B”, then it has been determined that the switch 18 is in the “auto” position and a block 508 selects an auto mode of operation. The integrated circuit 402 senses the positions of the switch 18 (and the switch 20, for that matter) by checking SW_READ, which, is noted above, is provided to the pin 19 of the IC 402.

Once the mode has been selected, a block 510 checks the position of the switch 20 in a fashion similar to the blocks 500-508 described above to determine the selected emission frequency. Once the emission frequency has been determined, a block 512 causes the IC 402 to develop the signals LOW_POWER, PWM, ENABLE1, and ENABLE2, in turn to cause the piezoelectric elements 430, 432 to be energized in accordance with the selected mode of operation and emission frequency. Specifically, a high frequency pulse-width modulated waveform having a frequency between about 130 kHz and about 165 kHz is provided as the control signal PWM, thereby causing the transistor Q2 to rapidly turn on and off, thereby causing high frequency alternating current power to be provided to the junction 420. When the piezoelectric element 430 is to be operated, a high state signal is provided as the signal ENABLE1 thereby turning on the transistor Q3. When the piezoelectric element 432 is to be operated, a high state signal is provided as the signal ENABLE2 thereby turning on the transistor Q4.

When the battery voltage has dropped to a particular level of, for example, 0.8 volts, a high state signal is provided as the LOW_POWER signal, thereby turning off the transistor Q4 and preventing further energization of the piezoelectric elements 430, 432. This feature prevents the battery from being discharged to the point where it would leak and damage the device 10.

In summary, a user may operate the device 10 to emit a selected one of two different active materials for a particular period of time at a selected emission frequency, or may cause the unit to alternate between emissions of different active materials at a selected emission frequency.

### INDUSTRIAL APPLICABILITY

The diffusion device described in the present application can be used to automatically dispense multiple active materials over an extended period of time, with the added advantage that the frequency of dispersion and the mode of operation may be adjusted. The diffusion device 10 may be placed in any one of a number of different holders to suit the individual preference of the user and/or to disguise the true purpose of the device 10.

Numerous modifications will be apparent to those skilled in the art in view of the foregoing description. Accordingly, this description is to be construed as illustrative.

We claim:

1. A diffusion device, comprising:
a housing;
first and second containers disposed within the housing and having first and second wicks, respectively, extending therefrom and;
first and second active materials disposed in the first and second containers, respectively;
first and second piezoelectric elements disposed adjacent tips of the first and second wicks, respectively; and
a switch disposed on a top surface of the housing, wherein the switch is adapted to control the mode of operation of the device, wherein the device includes first, second, and third modes of operation and wherein in the first mode of operation, the device emits the first active material, in the second mode of operation, the device emits the second active material, and in the third mode of operation, the device alternates between emitting the first and second active materials.

2. The diffusion device of claim 1, wherein alternation between the first and second active materials comprises emitting the first active material for three hours, emitting the second active material for three hours, and repeating this pattern.

3. The diffusion device of claim 1, wherein alternation between the first and second active materials comprises emitting the first active material for twenty-four hours, emitting the second active material for twenty-four hours, and repeating this pattern.

4. The diffusion device of claim 2, wherein the diffusion device includes a second switch for selecting an intensity level for dispersion of the active material(s).

5. The diffusion device of claim 4, wherein the intensity level is determined by the time between active material emissions and wherein the time between active material emissions is between about 1 second and about 30 seconds.

6. The diffusion device of claim 5, wherein the device includes three different emission levels from which to select.

7. The diffusion device of claim 6, wherein the three different emission levels include time intervals between active material emissions of 9 seconds, 12 seconds, and 18 seconds.

8. The diffusion device of claim 5, wherein the device includes five different emission levels from which to select.

9. The diffusion device of claim 1, wherein the first and second active materials are different.

10. The diffusion device of claim 1, further including a battery for powering the diffusion device.

11. A combination, comprising:
a diffusion device including
a housing,
first and second containers disposed within the housing,
first and second wicks extending respectively from the first and second containers,
first and second active materials disposed in the first and second containers, respectively,
first and second piezoelectric elements disposed adjacent tips of the first and second wicks, respectively, for dispensing the first and second active materials, respectively; and
a holder having a cavity disposed therein, wherein the diffusion device is placed in the cavity to create a decorative object.

12. The combination of claim 11, wherein the decorative holder is formed in the shape of a leaf. (to be inserted at a later date when the designs are further developed; dependent claim for each design going into production).

13. The combination of claim 11, wherein the diffusion device is battery operated.

14. The combination of claim 11, wherein the diffusion device is retained in the holder by an interference fit.

15. A method of dispensing first and second active materials from a diffusion device, the method comprising the steps of:
inserting a battery into the diffusion device;
selecting an intensity level for dispersion of the active material(s), wherein the intensity level is determined by the time between active material emissions; and
selecting from one of the three different modes of operation including
emitting the first active material;
emitting the second active material; and
alternating between emission of the first and second active materials based on a predetermined emission timing.

16. A battery-operated diffusion device, comprising:
a housing;
a battery disposed within the housing for providing power to the diffusion device;
first and second containers disposed within the housing and having first and second wicks, respectively, extending therefrom and;
first and second active materials disposed in the first and second containers, respectively;
first and second piezoelectric elements disposed adjacent tips of the first and second wicks, respectively;
a first switch disposed on a top surface of the housing, wherein the first switch is adapted to control the intensity of diffusion; and
a second switch disposed on a top surface of the housing, wherein the second switch is adapted to control the mode of operation of the device and includes three different modes of operation for operating the diffusion device.

17. The battery-operated diffusion device of claim 16, wherein the device includes at least mode of operation selected from the group of rotating between emission of the first and second active materials, gradually increasing and decreasing emission of an active material, increasing and decreasing the emission of an active material at predetermined time periods, and periodically discontinuing emission of an active material for a predetermined time period.

18. The battery-operated diffusion device of claim 16, in combination with a holder.

19. The battery-operated diffusion device of claim 18, wherein the holder includes a cavity disposed therein and wherein the diffusion device is disposed with the cavity to create a decorative object.

20. The battery-operated diffusion device of claim 19, wherein the holder is in the shape of a leaf.
